(19) United States
(12) Patent Application Publication
(10) Pub. No.: US 2015/0289901 A1
(43) Pub. Date: Oct. 15, 2015

(54) APPARATUS FOR CREATING PORTAL SITES USING A BLADE AND NEEDLE

(71) Applicants: Aman Shabi Khan, Daly City, CA (US); Danny Kay PA-C, Daly City, CA (US)

(72) Inventors: Aman Shabi Khan, Daly City, CA (US); Danny Kay PA-C, Daly City, CA (US)

(21) Appl. No.: 14/251,848

(22) Filed: Apr. 14, 2014

(51) Int. Cl. A61B 17/3209; A61B 17/34

(52) U.S. Cl. CPC A61B 17/3209; A61B 17/3496; A61B 17/3478

(57) ABSTRACT

An apparatus comprises a barrel comprising an interior and an open end. The barrel is configured for engaging a needle along a longitudinal length where the engaged needle extends a distance past the open end. A rod mechanism is disposed within the interior for longitudinal movement and is configured for joining a knife having a blade end where the joined knife is disposed within the interior with the rod mechanism in a retracted position. The blade end extends a distance past the open end with the rod mechanism in an extended position. A spring mechanism engages with the rod mechanism and urges the rod mechanism to be in the retracted position. A plunger device engages with the rod mechanism and applies a force to the rod mechanism to compress the spring mechanism and move the rod mechanism to the extended position.

### CROSS-REFERENCE TO RELATED APPLICATIONS

Not applicable.

### RELATED CO-PENDING U.S. PATENT APPLICATIONS

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

One or more embodiments of the invention generally relate to creating portal sites. More particularly, the invention relates to creating portal sites using a blade and needle.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Current devices for creating portal sites may have a variety of drawbacks, including, without limitation, a lack of safety precautions to prevent cutting too deeply, manual blade retraction, and unfamiliar mechanics.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. One such example of the prior art shows a surgical system for penetrating and cutting tissue which has a handle provided with a spring or friction slide to advance or retract a sleeve containing a knife By way of educational background, another aspect of the prior art generally useful to be aware of teaches of a sliding knife and needle assembly for creating a percutaneous incision or portal in a living body which comprises an elongate hollow rectangular handle having a closed first end, an open second end and a slot extending along one side, with the handle defining a first central longitudinal axis. Further, by way of educational background, another aspect of the prior art generally useful to be aware of shows a method of performing arthroscopic surgery which includes inserting a cannulated needle into a joint area of the body, inserting a guidewire through the needle, removing the needle, and inserting an arthroscopy knife into the joint area via the use of the guidewire. Finally, another aspect of the prior art discloses of a safety surgical instrument having a sheath, a handle, a surgical element such as a blade or needle, and a manner for moving the surgical element out of and into the sheath by rotary and longitudinal movement of the handle. However, these solutions may not provide users with a device having easy-to-use components which may allow users to safely and effectively create portal sites. A solution which did so would be desirable.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is an illustration of an exemplary device for creating portal sites, in accordance with an embodiment of the present invention;

FIG. 2 is an illustration of an exemplary device for creating portal sites in a recoiled position;

FIG. 3 is an illustration of an exemplary method for creating portal sites, in accordance with an embodiment of the present invention;

FIG. 4 is an illustration of an exemplary method for augmentation for a central line placement, in accordance with an embodiment of the present invention; and

FIG. 5 is an illustration of an exemplary device for creating portal sites being used by a robotic arm, in accordance with an embodiment of the present invention.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

Headings provided herein are for convenience and are not to be taken as limiting the disclosure in any way.

The enumerated listing of items does not imply that any or all of the items are mutually exclusive, unless expressly specified otherwise.

The terms “a”, “an” and “the” mean “one or more”, unless expressly specified otherwise.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

Some embodiments of the present invention may provide means and/or methods for creating portal sites. Other embodiments may provide augmentation for a central line placement, cardiac catheterization, or any location where an incision on skin needs to be created followed by needle placement. Many embodiments may comprise blades and/or needles for use in various procedures, including, without limitation, medical procedures. In some embodiments, a blade may attach to a needle. In a non-limiting example, an “11 blade” may attach to a spinal needle. As a non-limiting example, spinal needles may come in various sizes 3.5 in, 4.1 in, 4.8 in, 5.5 in, 5.9 in. Any size may be utilized with the spinal knife depending on the user preferences. In other embodiments, an embodiment device may have blade and needle components which do not attach to each other.

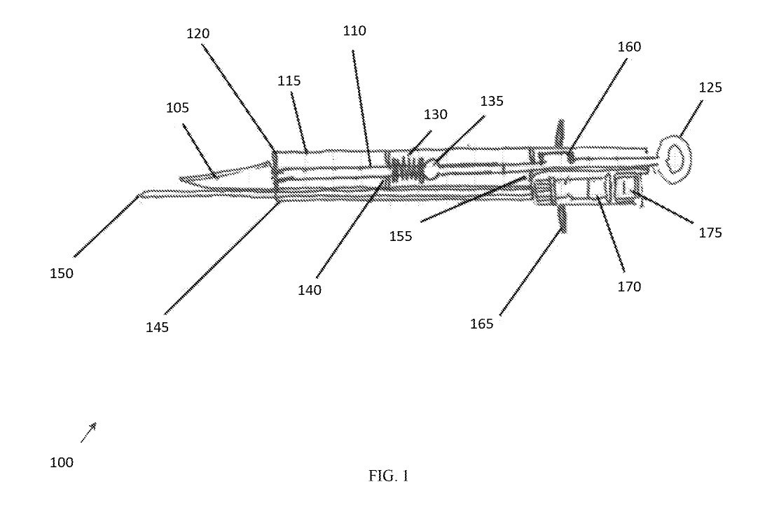

FIG. 1 is an illustration of an exemplary device for creating portal sites, in accordance with an embodiment of the present invention. In the present embodiment, a blade 105 may attach to a rod 110, and the blade 105 and rod 110 may be housed within a barrel 115. In some embodiments, the barrel 115 may be any shape and/or size. In a non-limiting example, a barrel 115 may be rectangular in shape. In the present embodiment, the barrel 115 may have an opening, or barrel head 120, where the blade 105 may protrude. Further, in the present embodiment, a plunger 125 at an end of the barrel 115 may control the blade 105. In some embodiments, the plunger 125 may be any size and/or shape. In a non-limiting example, a plunger 125 may have a flat end or handle. In some embodiments, the blade 105 may be retractable and controllable by a compression spring mechanism 130, 135. In the present embodiment, stoppers 140 may hold a spring 130 in place to prevent advancement and/or recoil of the blade 105. In some embodiments, stoppers 140 may be fixed in place. In alternative embodiments, stoppers 140 may be adjustable to allow users to determine a desired depth for each use. Further, in the present embodiment, the barrel 115 may also contain a needle sleeve 145 which may house a needle 150. In some embodiments, the barrel 115 may prevent the needle 150 from advancing beyond a desired depth, or point.

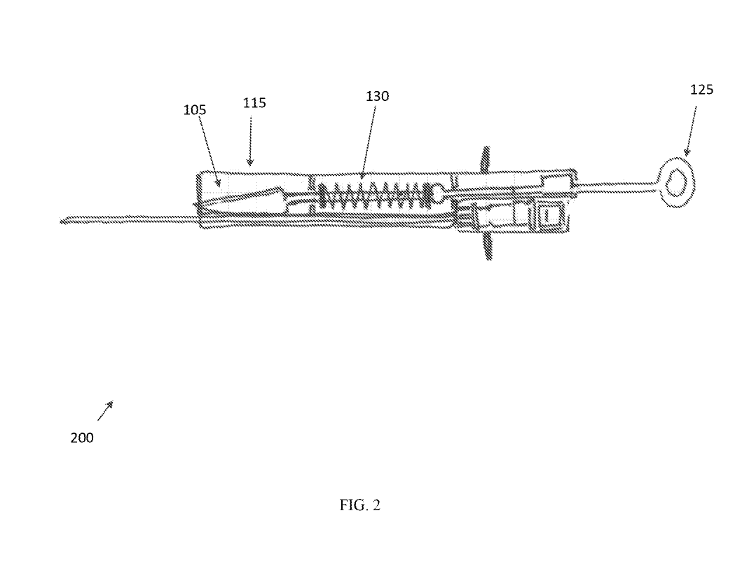

FIG. 2 is an illustration of an exemplary device for creating portal sites in a recoiled position. In the present embodiment, the blade 105 may come to rest within the barrel 115 when a sufficient amount of pressure is not applied to the plunger 125. In some embodiments, a non-limiting pressure range may be on the order of 0.2 to 0.4 pounds per square inch. In the present embodiment, the spring 130 may be in a relaxed or uncoiled state when a sufficient amount of pressure is not applied to the plunger 125.

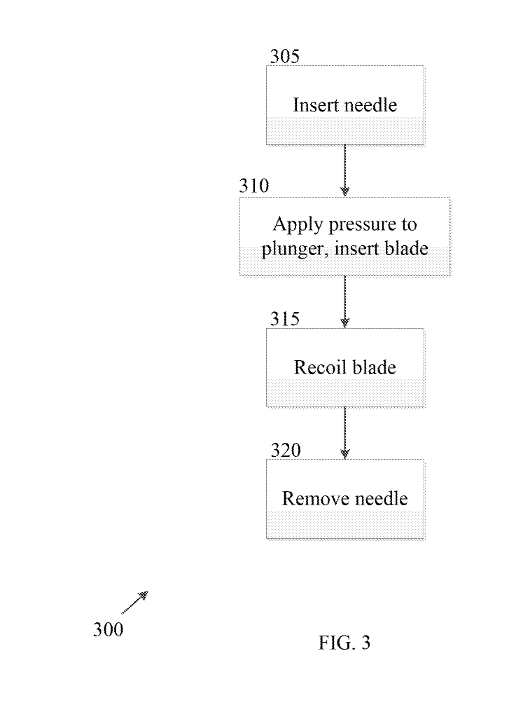

FIG. 3 is an illustration of an exemplary method for creating portal sites, in accordance with an embodiment of the present invention. In the present embodiment, a user may insert a needle 150 into a potential portal site of a patient in a step 305. In a non-limiting example, a user may insert the needle 150 into a joint. In the present embodiment, a user may apply pressure to a plunger 125 to advance a blade into a patient in a step 310. In some embodiments, a system of components may allow a user to safely control the blade. In a non-limiting example, compression spring mechanisms 130, 135 and stoppers 140 may assist a user by providing counter-pressure and by preventing the blade 105 from advancing beyond a desired point. In the present embodiment, a user may allow a blade 105 to recoil by releasing pressure from a plunger 125 in a step 315. Further, in the present embodiment, a user may remove the needle 150 in a step 320. In some embodiments, the user may skip step 315 and in step 320 remove the needle 150 with blade 105 still protruding.

In some embodiments, various components may be removable and/or interchangeable. In a non-limiting example, a first blade 105 may be switched out for a second blade 105. In the present non-limiting example, the first blade 105 may be a different size than the second blade 105. In some embodiments, a barrel 115 may be designed to receive a variety of types of blades 105, including, without limitation, blades 105 designed by other makers.

Some embodiments may incorporate one or more “stoppers” which may prevent a blade and/or a needle from penetrating too deep. In one embodiment, a first stopper 140 may keep a compression spring 130 in place, which may allow compression to occur when pressure is applied to a plunger 125. In the present embodiment, the first stopper 140 may block the spring 130 and a spring knot 135 from advancing. In one embodiment, a second stopper 155 may prevent a secondary knot 160 from advancing.

In some embodiments, a blade 105 may automatically retract. In one embodiment, a blade 105 may protrude from a barrel housing 115 when pressure is applied to a plunger 125, and the blade 105 may automatically retract when the pressure is released from the plunger 125. In some embodiments, a compression spring recoil system may allow a blade 105 to retract. In other embodiments, any suitable method for causing blade 105 retraction may be used, including, without limitation, a hydraulic recoil system. In alternative embodiments, a blade 105 may maintain any position without an external force by a user. In some of these alternative embodiments, various components, including, without limitation, a barrel 115 may apply frictional force to the blade 105 to maintain blade 105 position, and a user may pull on a plunger 125 to recoil the blade 105.

Some embodiments may allow users to create an incision in a single step while a needle 150 may be in place. In some of these embodiments, a blade 105 may protrude when pressure is applied to a plunger 125, which may create an incision/portal in a single step.

Many embodiments may allow for removal and/or replacement of a needle 150. Some of these embodiments may have a sleeve 145 which may house a needle 150, and the sleeve 145 may snap in place. In some of these embodiments, a user may remove the needle 150 from the sleeve 145 and use the needle 150 independently, and replace the needle 150 as desired. In some embodiments, a needle 150 may fit into a housing which also contains a blade 105. In alternative embodiments, a needle 150 may have a separate housing which may attach to an outer side of a blade 105 housing. In some of these alternative embodiments, the blade 105 and needle 150 may be separated by outer sides of one or more housing. In some alternative embodiments, an embodiment device may have one or more switches which a user may press to release device components. In a non-limiting example, a switch on an outer side of a barrel 115 may, when pressed, release a needle 150 from being fixed in place, and a user may then remove the needle 150. In some embodiments, components may be held in place by any suitable method, including, without limitation, frictional force, springs, or covers. In a non-limiting example, a cover may hold a blade 105 and/or needle 150 in place while secured to a device, and the blade 105 and/or needle 150 may be loose and/or removable when the cover is not secured to the device.

Some embodiments may incorporate familiar, easy-to-use mechanics. In a non-limiting example, a user may advance a blade 105 by applying pressure to a plunger 125, which may be mechanically similar to using a syringe.

In some embodiments, a finger flange 165 may provide counter-force against pressure applied to a plunger 125. The index and middle finger may direct backward pressure on the device as the thumb applies forward pressure on the plunger 125 directing the blade 105 out of the housing. This effectively provides counter-force against the thumb driving the plunger forward. Without the finger flange 165 the index and middle fingers would have the grip tightly on the housing of the device as pressure is applied to the plunger. In some of these embodiments, the finger flange 165 may be any shape and/or size. In a non-limiting example, a finger flange 165 may be circular or cylindrical.

In at least one embodiment, a needle base 170 may come into contact with a stylet base 175. In some embodiments, the stylet base 175 may allow for removal of a stylet. The stylet keeps the barrel of the needle 150 closed during a skin puncture. The stylet may be removed if the spinal needle is to be used with a syringe to inject.

Some embodiments may be composed of any suitable material. In a non-limiting example, an embodiment device may be composed entirely of metal with a needle 150 and blade 105 which users may replace. In another non-limiting example, all components may be composed of metal, excepting for a spring 130, blade 105, and needle 150.

Many embodiments may be split into separate components. In a non-limiting example, an embodiment device may have four separate components: a blade 105, a recoil plunger 125 mechanism, a needle 150, and an attachable finger flange 165.

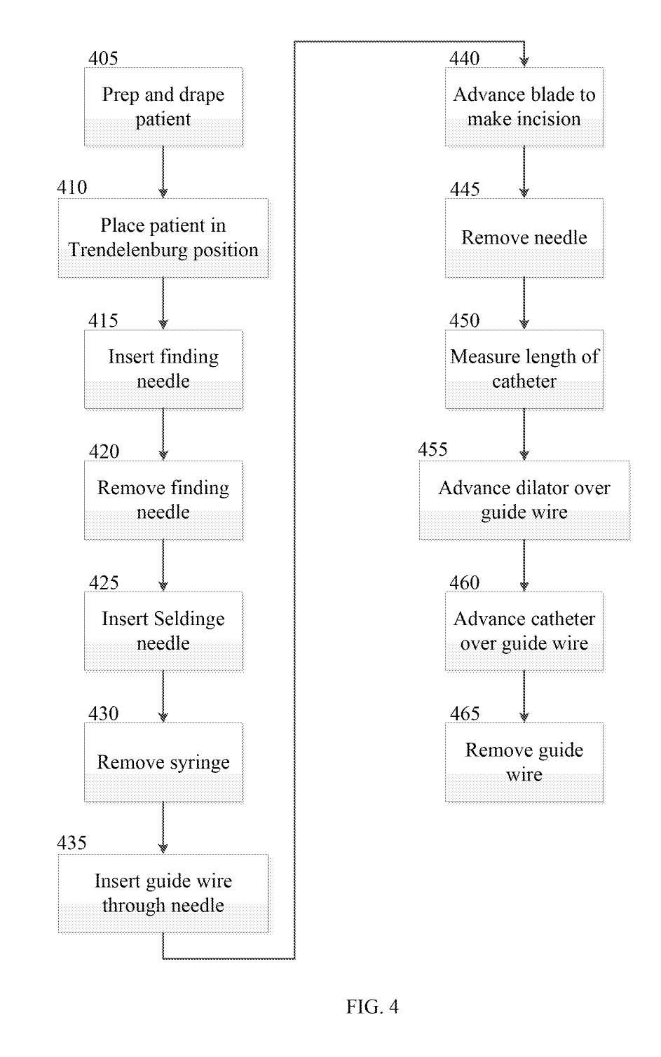

FIG. 4 is an illustration of an exemplary method for augmentation for a central line placement, in accordance with an embodiment of the present invention. In the present embodiment, when performing a central line placement a patient is sterilely prepped and draped in a step 405. The patient is placed in a Trendelenburg position with the head slightly turned left in a step 410. The entry point is at an apex of a triangle formed by the clavicle and medial and lateral heads of the sternocleidomastoid muscle. Other important structures to note are the external jugular vein and carotid artery. A finding needle is inserted at the apex of the triangle and slowly advanced, staying lateral to the carotid pulse in a step 415. Seeing a trickle of blood confirms entry in the jugular vein. The finding needle is withdrawn in a step 420, while the location and angle of entry is mentally noted. Next a Seldinger needle 150 with an attached syringe is re-inserted in a step 425. The syringe is then carefully removed from the needle a step 430 and a guide wire is fed through the Seldinger needle 150 until the mark is reached in a step 435. Then an 11 blade 105 is advanced to make a small incision where the wire enters the skin in a step 440. This will facilitate the passage of the catheter. The guide wire is held in place as the needle is carefully removed a step 445. The length of catheter is measured from the sternal notch to the needle entry point a step 450. A dilator is carefully advanced over the wire to dilate the soft tissue in a step 455. Next the central line is advanced over the wire in a step 460 and the guide wire is removed a step 465.

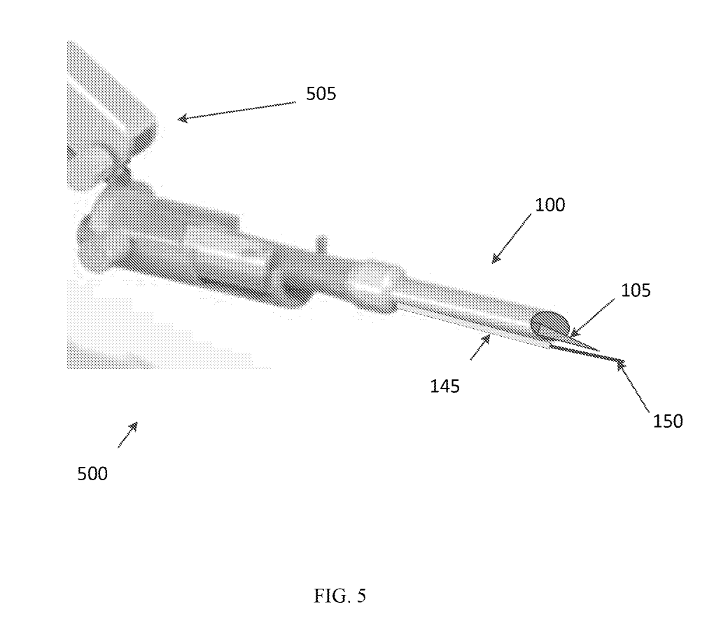

FIG. 5 is an illustration of an exemplary device for creating portal sites being used by a robotic arm, in accordance with an embodiment of the present invention. In the present embodiment, device 100, as exemplarily shown in FIG. 1 and FIG. 2, may be adapted for control by a robotic arm 505. In the present embodiment, the robotic arm may be remotely controlled to insert needle 150 and advance blade 105 to make an incision in accordance with the teachings of the present invention.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied. Thus, the present invention is not limited to any particular tangible means of implementation.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC §112 (1), all claims must be supported by sufficient disclosure in the present patent specification, and any material known to those skilled in the art need not be explicitly disclosed. However, 35 USC §112 (6) requires that structures corresponding to functional limitations interpreted under 35 USC §112 (6) must be explicitly disclosed in the patent specification. Moreover, the USPTO's Examination policy of initially treating and searching prior art under the broadest interpretation of a “mean for” claim limitation implies that the broadest initial search on 112(6) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC §112 (6) when such corresponding structures are not explicitly disclosed in the foregoing patent specification. Therefore, for any invention element(s)/structure(s) corresponding to functional claim limitation(s), in the below claims interpreted under 35 USC §112 (6), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC §112 (6) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC §112 (6), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC §112 (6). Applicant(s) note that all the identified documents above which are incorporated by reference to satisfy 35 USC §112 (6) necessarily have a filing and/or publication date prior to that of the instant application, and thus are valid prior documents to incorporated by reference in the instant application.

Having fully described at least one embodiment of the present invention, other equivalent or alternative methods of implementing creation of portal sites according to the present invention will be apparent to those skilled in the art. Various aspects of the invention have been described above by way of illustration, and the specific embodiments disclosed are not intended to limit the invention to the particular forms disclosed. The particular implementation of the creation of portal sites may vary depending upon the particular context or application. By way of example, and not limitation, the creation of portal sites described in the foregoing were principally directed to spinal surgery implementations; however, similar techniques may instead be applied to any kind of surgery, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The Abstract is provided to comply with 37 C.F.R. Section 1.72(b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

What is claimed is:

1. An apparatus comprising:
a barrel member comprising an interior space and at least one open end, said barrel member being further configured for engaging a needle device along a longitudinal length of said barrel member where the engaged needle device extends a first distance exteriorly past said one open end;
a rod mechanism being disposed within said interior space and being configured for longitudinal movement within said barrel member, said rod mechanism being further configured for joining a knife device having a blade end where the joined knife device is disposed within said interior space with the blade end interior to said open end with said rod mechanism in a retracted position, and the blade end extends a second distance exteriorly past said one open end with said rod mechanism in an extended position, said second distance being a portion of said first distance;
a compression spring mechanism being in engagement with said rod mechanism, said compression spring mechanism being configured for urging said rod mechanism to be in said retracted position within said barrel member; and
a plunger device being in engagement with said rod mechanism, said plunger device being configured for applying a user supplied force to said rod mechanism to compress said compression spring mechanism and move said rod mechanism to said extended position.

2. The apparatus as recited in claim 1, further comprising a needle sleeve joined to said barrel member, said needle sleeve being configured to house at least a portion of an engaged needle device.

3. The apparatus as recited in claim 1, further comprising a first stopper being disposed within said interior space, said first stopper being configured for stopping movement of said rod mechanism at said extended position and at said retracted position.

4. The apparatus as recited in claim 3, further comprising a second stopper being disposed within said interior space, said second stopper being configured for stopping movement of said rod mechanism at said retracted position.

5. The apparatus as recited in claim 1, further comprising at least one finger flange joined to said barrel member, said at least one finger flange being configured for applying a counter-force against pressure applied to said plunger device.

6. The apparatus as recited in claim 1, in which said rod mechanism further comprises a spring knot being configured for engaging said compression spring mechanism.

7. The apparatus as recited in claim 1, further comprising a knife device having a blade end for removably engaging said rod mechanism.

8. The apparatus as recited in claim 2, further comprising a needle device for removably engaging said needle sleeve.

9. An apparatus comprising:
means for engaging a needle device along a longitudinal length of said engaging means where the engaged needle device extends a first distance past an end of said engaging means;
means for moving longitudinally within said engaging means, said moving means being further configured for joining a knife device having a blade end where the joined knife device is disposed within said engaging means with the blade end interior to said engaging means with said moving means in a retracted position, and the blade end extends a second distance exteriorly past said engaging with said moving means in an extended position, said second distance being a portion of said first distance;
means for urging said moving means to be in said retracted position; and
means for applying a user supplied force to said moving means to compress said urging means and move said moving means to said extended position.

10. The apparatus as recited in claim 9, further comprising means for housing at least a portion of an engaged needle device.

11. The apparatus as recited in claim 9, further comprising a first means for stopping movement of said moving means at said extended position and at said retracted position.

12. The apparatus as recited in claim 11, further comprising a second means for stopping movement of said moving means at said retracted position.

13. The apparatus as recited in claim 9, further comprising means for applying a counter-force against pressure applied to said applying means.

14. The apparatus as recited in claim 9, further comprising a knife device having a blade end for removably engaging said moving means.

15. The apparatus as recited in claim 10, further comprising a needle device for removably engaging said housing means.

16. An apparatus comprising:
a barrel member comprising an interior space and at least one open end;
a needle sleeve joined to said barrel member, said needle sleeve being configured for housing at least a portion of an engaged needle device along a longitudinal length of said barrel member where the engaged needle device extends a first distance exteriorly past said one open end
a rod mechanism being disposed within said interior space and being configured for longitudinal movement within said barrel member, said rod mechanism being further configured for joining a knife device having a blade end where the joined knife device is disposed within said interior space with the blade end interior to said open end with said rod mechanism in a retracted position, and the blade end extends a second distance exteriorly past said one open end with said rod mechanism in an extended position, said second distance being a portion of said first distance;
a compression spring mechanism being in engagement with said rod mechanism, said compression spring mechanism being configured for urging said rod mechanism to be in said retracted position within said barrel member;
a plunger device being in engagement with said rod mechanism, said plunger device being configured for applying a user supplied force to said rod mechanism to compress said compression spring mechanism and move said rod mechanism to said extended position; and;
a knife device having a blade end for removably engaging said rod mechanism; and
a needle device for removably engaging said needle sleeve.

17. The apparatus as recited in claim 16, further comprising:
a first stopper being disposed within said interior space, said first stopper being configured for stopping movement of said rod mechanism at said extended position and at said retracted position;
a second stopper being disposed within said interior space, said second stopper being configured for stopping movement of said rod mechanism at said retracted position; and
at least one finger flange joined to said barrel member, said at least one finger flange being configured for applying a counter-force against pressure applied to said plunger device, in which said rod mechanism further comprises a spring knot being configured for engaging said compression spring mechanism.

18. The apparatus as recited in claim 16, in which said knife device further comprises a spinal knife.

19. The apparatus as recited in claim 18, in which said spinal knife further comprises an 11 blade.

20. The apparatus as recited in claim 16, in which said needle knife further comprises a spinal needle.
